(19) United States
(12) Patent Application Publication
(10) Pub. No.: US 2006/0188947 A1
(43) Pub. Date: Aug. 24, 2006

(54) Reagent for producing a protein chip

(76) Inventors: Yaeta Endo, Ehime (JP); Tatsuya Sawasaki, Ehime (JP)

Correspondence Address: KILYK & BOWERSOX, P.L.L.C., 400 HOLIDAY COURT, SUITE 102, WARRENTON, VA, 20186, US

(21) Appl. No.: 10/565,494

(22) PCT Filed: Aug. 5, 2004
(86) PCT No.: PCT/JP04/11567

§ 371 Date: Jan. 20, 2006

Related U.S. Application Data
(60) Provisional application No. 60/542,201, filed on Feb. 5, 2004.

(30) Foreign Application Priority Data
Aug. 7, 2003 (JP) 2003-288859

(51) Int. Cl. G01N 33/53; C12P 21/06

(52) U.S. Cl. 435/7.5; 435/68.1

(57) ABSTRACT

An object of the present invention is to provide a reagent for protein synthesis by a simple operation which is advantageous in a cell-free protein synthesis in many samples in a test using an aimed (specific) protein or a high throughput analysis using a protein chip, etc. and also to provide a method for the synthesis of protein using the same. Further objects are to provide a protein library using the above-mentioned reagent, a kit for preparing a protein chip, a kit for testing an aimed (specific) protein, etc.

The present invention claims the benefit of priority from Japanese Patent Application Nos. 2003-288859 and U.S. Provisional Patent Application No. 60/542,201, of which full contents are incorporated herein by reference.

### TECHNICAL FIELD

The present invention relates to a protein chip reagent using a cell-free protein synthesis system, to a kit for protein chip reagent using a cell-free protein synthesis system and to a test method using the reagent. More particularly, it relates to a reagent and a kit for a test and to a test method wherein a reagent manufactured by freeze-drying of a translation reaction solution where a substance necessary for protein synthesis, a translation template, a stabilizer, etc. are added to a solution containing a cell extract for a cell-free protein synthesis is used so as to express the protein upon each use and interaction with the said protein is utilized.

### BACKGROUND ART

Synthetic reaction of protein carried out in cells proceeds with such steps that, firstly, information in DNA having genetic information is transcribed to mRNA and ribosome translates the information in RNA whereupon protein is synthesized. With regard to a method for the protein synthesis in cells outside the living body such as in vitro, there has been briskly carried out at present a study for a cell-free protein synthesis in vitro where ribosome and other components necessary for protein synthesis are extracted from living body (in the present specification, that may be referred to as “cell extract for a cell-free protein synthesis”) and used therefor (Patent Document 1, Patent Document 2, Patent Document 3, Patent Document 4 and Patent Document 5).

Such a solution containing cell extract for a cell-free protein synthesis and a protein synthesis reaction solution in which components necessary for translation except translation template and enzyme are added to the said extract-containing solution (hereinafter, that may be referred to as “a cell extract-containing solution of a ready-made type”) are unstable at ambient temperature and have been able to be stably preserved only at an extremely low temperature of not higher than −80° C. However, a cell-free protein synthesis system has a property which is as good as living cells in view of accuracy and speed of the translation reaction and is a useful method where a specific protein is able to be prepared without conducting complicated purifying steps. Therefore, in order to apply the said synthesis system to industry much more, it is necessary not only to raise the synthetic efficiency but also to provide the above solution containing a cell extract for synthesis and the above-mentioned solution containing a cell extract of a ready-made type keeping their high quality in a stable manner.

Until now, for the cell-free protein synthesis, it has been necessary that each of various components such as a cell extract-containing solution, amino acids, ATP, GTP, potassium and magnesium is kept in a stable state and, prior to the start of the protein synthesis reaction, they are mixed in an optimum ratio. In this method, troublesome operations are necessary in addition to the preservation method and, therefore, there is a big difficulty when numbers of synthetic samples are many. In addition, such a conventional method is a neck for constituting a fully automated system for the high throughput protein synthesis.

The present inventors have previously proposed a method for the manufacture of a preparation of a solution containing a cell extract for protein synthesis or a solution containing a cell extract of a ready-made type by means of freeze-drying (Patent Document 6). However, there is a problem in the said preparation that dissolving or the like happens during the freeze-drying step and, as a result, quality of the said preparation is deteriorated. Deterioration of quality means that, when water is added to the said preparation, the preparation is not completely dissolved and the synthetic activity in the protein synthesis reaction using the same is deteriorated as well.

In order to solve the above problems, there has been a proposal for a freeze-dried preparation showing an improved preservability (conservation) where polyhydric alcohol such as inositol is added for the stabilization under freezing preservation and amount of a deliquescent substance is lowered (Patent Document 7). However, since it is a product where a solution containing a cell-free extract is made into a preparation, it is necessary to further add a substance necessary for translation reaction and a translation template in handling many samples. Accordingly, the operation is troublesome and, in addition, mRNA which is a translation template is very unstable and is to be prepared upon each use whereby that is hardly said to be a preparation which is suitable for making in a high throughput one.

Further, in a conventional test method for an interacting substance with a specific protein, substances necessary for translation reaction and a specific translation template are added before expression of protein as mentioned above in order to analyze the interaction of many samples with the specific protein whereby the operation is troublesome. Furthermore, mRNA which is a translation template is very unstable and is to be prepared upon each use whereby no method where many samples are able to be tested quickly and easily has been established yet.

On the other hand, a proteome analysis where analysis is carried out using all proteins expressed in living body as objects is roughly classified into an expression proteomics and a functional (interacting) proteomics.

An expression proteomics is a means for a full analysis of a specific protein in such a respect that in which place and to what extent it is expressed in a living body while a functional proteomics is a means for a full analysis of a specific protein in such a respect that with what molecule it conducts an interaction. Up to now, such analyses have been carried out by means of a two-dimensional electrophoresis, a 2-hybrid method using yeast, a surface plasmon method, a phage display method, etc. but there are problems in terms of analytical time, sensitivity, pseudo-positivity reaction and quantity of date required for analysis and, accordingly, development for a protein chip where protein is accumulated in a high density has now been carried out.

With regard to a protein chip, there have been developed a method where serum or the like is contacted to antibody chip and the antigen-antibody reaction resulted on the said chip is detected by a solid-phase enzyme immunoassay (hereinafter, it may be referred to as “ELISA method”), a surface-enhanced laser desorption/ionization (SELDI) protein chip system where chip in which protein is made into a solid phase and MALDI-TOF MS (matrix-assisted laser desorption ionization/time-of-flight mass spectrometer) (Non-patent Document 1, Non-patent Document 2) are combined, etc.

In those analysis means, quality of the protein chip which greatly affects the reproducibility, etc. is a big problem. Since protein has an inherent function only when its three-dimensional structure is retained, there has been developed a surface chemical technique for a substrate in order to prevent its three-dimensional structure upon being solidified. In addition, since protein is very unstable as compared with DNA, etc. and its synthetic operation is troublesome as well, it is difficult that many samples are handled together with keeping their functions. Further, the prepared chip is to be used within about three day and its handling is inconvenient.

Patent Document 1: Japanese Laid-Open Patent Publication Hei-6/98790

Patent Document 2: Japanese Laid-Open Patent Publication Hei-6/225783

Patent Document 3: Japanese Laid-Open Patent Publication Hei-7/194

Patent Document 4: Japanese Laid-Open Patent Publication Hei-9/291

Patent Document 5: Japanese Laid-Open Patent Publication Hei-7/147992

Patent Document 6: Japanese Laid-Open Patent Publication 2000/316594

Patent Document 7: Japanese Laid-Open Patent Publication 2002/125693

Non-patent Document 1: Koster, H., et al., Nature Biotechnol., 14, 1123-1128 (1996)

Non-patent Document 2: Griffin, T. J., et al., Nature Biotechnol., 15, 1368-1372 (1997)

### DISCLOSURE OF INVENTION

An object of the present invention is to provide a protein chip reagent using a cell-free protein synthesis system by a simple operation which is particularly advantageous in a cell-free protein synthesis in many samples in a test using a specific protein or a high throughput analysis using the above-mentioned protein chip, etc. and also to provide a kit using the same. Further objects are to provide a kit for preparing a protein library or a protein chip using the above reagent and also to provide a test method for interacting substances.

In order to solve the above-mentioned problems, the present inventors have repeatedly carried out intensive studies and, as a result, they have found that a specific protein is synthesized in a translation reaction solution of a microtiter plate when a translation reaction solution in which substances necessary for protein synthesis, translation template and stabilizer are added to a solution containing a cell extract for a cell-free protein synthesis is subjected to a dry-freezing in a well of a microtiter plate, calcium acetate is added to the said well to dissolve the reagent and then a translation reaction is carried out by layering a solution containing substrate, energy source, etc. used for the cell-free protein synthesis, energy source thereupon. The present invention has been achieved on the basis of such a finding.

The present invention comprises the followings.

“1. A protein chip reagent utilizing a cell-free protein synthesis system which comprises the following elements.

a: a solution containing cell extract for a cell-free protein synthesis comprising a wheat embryo extract wherefrom endosperm components and low-molecular protein synthesis inhibitors are substantially removed is added to each different well of a container which is partitioned in plural sections;

b: at least the substances necessary for protein synthesis containing substrate and energy source and a specific translation template are added to the well mentioned in “a”; and

c: the solution in the well mentioned in “b” is freeze-dried preparation (by freeze-drying).

2. A protein chip reagent utilizing a cell-free protein synthesis system which comprises the following elements.

a: a solution containing cell extract for a cell-free protein synthesis comprising a wheat embryo extract wherefrom endosperm components and low-molecular protein synthesis inhibitors are substantially removed is added to each different well of a container which is partitioned in plural sections;

b: at least the substances necessary for protein synthesis containing substrate and energy source and a specific translation template are added to the well mentioned in “a”;

c: the solution in the well mentioned in “b” is freeze-dried preparation (by freeze-drying); and

d: amount of a deliquescent substance in the freeze-dried preparation in the well mentioned in “c” is 0.01 part by weight or less to 1 part by weight of the protein in the said freeze-dried preparation.

3. A protein chip reagent utilizing a cell-free protein synthesis system which comprises the following elements.

a: a solution containing cell extract for a cell-free protein synthesis comprising a wheat embryo extract wherefrom endosperm components and low-molecular protein synthesis inhibitors are substantially removed is added to each different well of a container which is partitioned in plural sections;

b: at least the substances necessary for protein synthesis containing substrate and energy source and a specific translation template are added to the well mentioned in “a”;

c: the solution in the well mentioned in “b” is freeze-dried preparation (by freeze-drying);

d: amount of a deliquescent substance in the freeze-dried preparation in the well mentioned in “c” is 0.01 part by weight or less to 1 part by weight of the protein in the said freeze-dried preparation; and

e: different kind type of translation template is contained in each the solution mentioned in “b” and makes two or more kinds of proteins synthesizable in each different well of the container which is partitioned in plural sections.

4. A protein chip reagent utilizing a cell-free protein synthesis system which comprises the following elements.

a: a solution containing cell extract for a cell-free protein synthesis comprising a wheat embryo extract wherefrom endosperm components and low-molecular protein synthesis inhibitors are substantially removed is added to each different well of a container which is partitioned in plural sections;

b: at least the substances necessary for protein synthesis containing substrate and energy source and a specific translation template are added to the well mentioned in “a”;

c: the solution in the well mentioned in “b” is freeze-dried preparation (by freeze-drying);

d: amount of a deliquescent substance in the freeze-dried preparation in the well mentioned in “c” is 0.01 part by weight or less to 1 part by weight of the protein in the said freeze-dried preparation;

e: different kind type of translation template is contained in each the solution mentioned in “b” and makes two or more kinds of proteins synthesizable in each different well of the container which is partitioned in plural sections; and

f: protein synthesized from the translation template is modified for fixation and is also coated with a substance having affinity to a substance added by the said modification for fixation to a surface in the well and/or a carrier in the well.

5. The protein chip reagent utilizing a cell-free protein synthesis system according to the above 4, wherein the modification for fixation is at least one which is selected from making into avidin, biotin, streptoavidin and His tag.

6. A kit for a cell-free protein synthesis containing the protein chip reagent utilizing a cell-free protein synthesis system mentioned in any of the above 1 to 5.

7. A test method for an interacting substance with a specific protein translated from a specific translation template containing the following elements where a reagent mentioned in any of the above 1 to 5 is used.

(1) a protein chip reagent utilizing a cell-free protein synthesis system is dissolved upon each use;

(2) after dissolving, conditions for a protein translation reaction are regulated to synthesize a specific protein;

(3) a substance to be detected is added and it is confirmed whether the interaction with the specific protein which is synthesized upon each use takes place; and

(4) the interacted substance is judged either qualitatively or quantitatively using a marker.”

### BRIEF DESCRIPTION OF DRAWINGS

FIG. 1: It is a graph showing a synthesized amount of GFP protein where a regent of the present invention for a cell-free protein synthesis was used. In the graph, ● is a synthesized amount of GFP protein where the translation template was added and freeze-dried while ◯ is a synthesized amount of GFP protein where the translation reaction solution was not freeze-dried and the translation template was also added later.

FIG. 2: It is electrophoretic pictures showing protein contained in the translation reaction solution by an SDS-PAGE in a synthesis system using a reagent of the present invention for a cell-free protein synthesis. A: Reaction products where the translation reaction solution was not freeze-dried and the translation template was also added later. B: Reaction products where the translation template was added and freeze-dried.

FIG. 3: It is a radioautographic picture showing the protein contained in the translation reaction solution in a synthesis system using the reagent for a cell-free protein synthesis containing 96 kinds of different translation templates.

### DETAILED DESCRIPTION OF INVENTION

(1) Preparation of a Solution Containing a Cell Extract for Cell-Free Protein Synthesis

With regard to a solution containing a cell extract for a cell-free protein synthesis used in the present invention, a wheat embryo extract having a protein synthesizing ability in a cell-free protein synthesis system is optimum. Here, a cell-free protein synthesis system means a method where components including ribosome which is a protein translation device being furnished in the cell are extracted from living body and then transcription or translation template, nucleic acid and amino acid to be used as a substrate, energy source, various ions, buffer and other effective factors are added followed by conducting in vitro. In the above, there is a system where RNA is used as a template (hereinafter, that may be referred to as “cell-free translation system”) and a system where DNA is used and enzymes necessary for the transcription such as RNA polymerase are further added to conduct the reaction (hereinafter, that may be referred to as “cell-free transcription/translation system”). The cell-free protein synthesis system of the present invention includes both of the above cell-free translation system and cell-free transcription/translation system.

With regard to the solution containing a cell extract used in the present invention, specifically used ones are those prepared from microbes such as Escherichia coli, embryo of plant seeds and reticulocyte mammals such as rabbits, etc. With regard to the solution containing a cell extract, commercially available one may be used or it may be prepared from the above microbe, embryo, reticulocyte, etc. by a method known per se or, more particularly in that prepared from microbes such as Escherichia coli, according to a method mentioned in Pratt, J. M., et al., Transcription and Translation, Hames, 179-209, B. D. & Higgins, S. J., eds., IRL Press, Oxford (1984).

Examples of the commercially available solution containing a cell extract are as follows. With regard to that derived from Escherichia coli, they are E. coli S30 Extract System (manufactured by Promega) and RTS 500 Rapid Translation System (manufactured by Roche) and, with regard to that derived from rabbit reticulocytes, they are Rabbit Reticulocyte Lysate System (manufactured by Promega), etc. Further, with regard to that derived from wheat embryo which is a particularly preferred wheat embryo extract, its example is Proteios™ (manufactured by Toyobo). With regard to plant seeds, those of plants of Gramineae such as wheat, barley, rice and corn are preferred. As to the solution containing a cell extract according to the present invention, that where a wheat embryo extract solution is used is adequate.

As to a method for preparing a wheat embryo extract solution, a method mentioned, for example, in Johnston, F. B., et al., Nature, 179, 160-161 (1957) is used as a method for isolation of wheat embryo while, as an extracting method for a solution containing a cell extract from isolated embryo, a method mentioned, for example, in Erickson, A. H., et al., (1996) Meth. in Enzymol., 96, 38-50, etc. is used.

In an embryo extract which is adequately utilized in the present invention, the endosperm containing the substances which suppresses the protein synthesis function (the substances such as tritin, thionine and ribonuclease which act on mRNA, tRNA, translation protein factor, ribosome, etc. and suppress their functions) contained or retained in the material cell per se is removed almost completely and purified. Here, the expression that the endosperm is removed almost completely and purified stands for an embryo extract wherefrom an endosperm part is removed to such an extent that ribosome is not substantially deadenylated while the expression that ribosome is not substantially deadenylated means that degree of deadenylation of ribosome is less than 7% or, preferably, 1% or less.

The above-mentioned embryo extract contains proteins which are derived from the solution containing the cell extract or are separately added upon necessity. Although there is no particular limitation for the adding amount thereof, it is preferably 1 to 10% by weight or, more preferably, 2.5 to 5% by weight to the total composition in the case of a composition before the freeze-drying in view of stability upon preservation under the freeze-dried state or of usability and, in the case of a freeze-dried composition after the freeze-drying, it is preferably 10 to 90% by weight or, more preferably, 25 to 70% by weight of the total freeze-dried composition. Incidentally, the protein content used here is that which is calculated by measuring the absorptions (260, 280 and 320 nm).

(2) Decreasing the Deliquescent Substance from the Solution Containing a Cell Extract

In the above solution containing a cell extract, a solvent used for the extraction or a buffer used for gel filtration after extraction contains deliquescent substances such as potassium acetate and magnesium acetate. Therefore, when a translation reaction solution is prepared using the said cell extract-containing solution and is directly made into a dry preparation, there is a problem that dissolving or the like happens in a dry-freezing step and, as a result, quality of the said preparation is deteriorated. Deterioration of the quality means that, when water is added to the said preparation, the preparation is not completely dissolved and synthetic activity in the protein synthesis reaction using it is also deteriorated.

Accordingly, after the freeze-drying, concentration of the deliquescent substances contained in the said cell extract-containing solution is decreased within such an extent that quality of the preparation is not affected. Specific method for decreasing the deliquescent substances includes a gel filtration using a gel carrier which is previously equilibrated with a solution containing a decreased amount of or no deliquescent substances, a dialysis method, etc. By the method as such, a decrease is conducted to such an extent that the final concentration of the deliquescent substances in the finally prepared translation reaction solution is made not more than 60 mM. To be more specific, when a solution containing a wheat embryo extract is used, concentration of potassium acetate contained in the finally prepared translation reaction solution is decreased to not more than 60 mM or, preferably, not more than 50 mM.

Further, the amount of the substance showing deliquescence (deliquescent substance) which does not deteriorate the preservation stability under the freeze-dried state in the freeze-dried preparation is preferably not more than 0.01 part by weight or, particularly preferably, not more than 0.005 part by weight to 1 part by weight of protein contained in the said freeze-dried preparation.

Decreasing of the deliquescent substance may be carried out after preparing the solution containing the cell extract or may be carried out in any of the steps until preparation of the solution containing the cell extract.

The solution containing a cell extract may contaminate microbes, particularly spores of fungi and it is preferred to remove such microbes. Growth of the microbe may be sometimes noted particularly in a cell-free protein synthesis reaction for a long period (one day or longer) and, therefore, it is important to inhibit that. Although there is no particular limitation for the means of removing the microbes, it is preferred to use a filtration sterilization filter. With regard to the pore size of the filter, there is no particular limitation so far as it is a size by which microbes having a possibility of contamination are able to be removed and, usually, it is 0.1 to 1 micrometer or, preferably, 0.2 to 0.5 micrometer.

Further, when a step of removal of low-molecular synthesis inhibitors and/or a step of decreasing the concentration of reducing agent are/is added in any stage of the preparing process of the solution containing a cell extract, it is possible to give a solution containing a cell extract for conducting a cell-free protein synthesis having a specific effect.

(3) Method for Removal of Low-Molecular Synthesis Inhibitors from a Solution Containing a Cell Extract

The solution containing a cell extract contains low-molecular synthesis inhibitors having an activity of inhibiting the protein synthesis (hereinafter, that may be referred to as “low-molecular synthesis inhibitors”) and, when they are removed, it is possible to give a solution containing a cell extract having a high protein synthesis activity. To be more specific, low-molecular synthesis inhibitors are fractionated and removed by means of difference in molecular weights from the constituting components of the solution containing a cell extract. The low-molecular synthesis inhibitors are able to be fractionated as molecules having molecular weights of not more than the smallest one among the factors necessary for synthesis of protein contained in the solution containing a cell extract. To be specific, they are able to be fractionated and removed as those having molecular weights of not more than 50,000 to 14,000 and, preferably, not more than 14,000.

With regard to a method for removal of low-molecular synthesis inhibitors from a solution containing a cell extract, a commonly used method which has been known per se is used and, to be specific, a method by dialysis by way of a permeable membrane, a gel filtration method and an ultrafiltration method may be exemplified. Among them, a method by dialysis is preferred in view of easiness in providing a substance to an inner liquid for the dialysis.

With regard to a permeable membrane used for a removing operation of the low-molecular synthesis inhibitors by means of dialysis, that having a removing molecular weight of 50,000 to 12,000 is exemplified. To be specific, a regenerated cellulose membrane having a removing molecular weight of 12,000 to 14,000 (manufactured by Viskase Sales, Chicago), a Spectra/Pore 6 having a removing molecular weight of 50,000 (manufactured by Spectrum Laboratories Inc., CA, U.S.A.), etc. are preferably used. An appropriate amount of a solution containing a cell extract or the like is placed in such a permeable membrane and dialysis is carried out by a conventional method. With regard to the time for the dialysis, about 30 minutes to 24 hours is preferred.

When insoluble components are produced in the solution containing a cell extract in conducting the removal of the low-molecular synthesis inhibitors, the production as such is inhibited (hereinafter, that may be referred to as “stabilization of a solution containing a cell extract”) whereby it is possible to enhance the protein synthesis activity of the finally-prepared cell extract-containing solution or translation reaction solution. An example of the specific method for stabilization of the cell extract-containing solution or translation reaction solution is that, in conducting the removal of the above-mentioned low-molecular synthesis inhibitors, the cell extract-containing solution or translation reaction solution is made as a solution containing at least a high-energy phosphoric acid compound such as ATP or GTP (hereinafter, that may be referred to as “stabilizing component”). ATP is preferably used as a high-energy phosphoric acid compound. In addition, that is preferably carried out in a solution containing ATP and GTP or, more preferably, ATP, GTP and 20 kinds of amino acids.

Those components may be subjected to a removing step for the low-molecular inhibitors after adding the stabilizing components thereto followed by incubating previously or, when a dialysis method is used for the removal of the low-molecular synthesis inhibitors, it is also possible that the stabilizing components are also added to the outer solution for dialysis followed by dialyzing whereupon the low-molecular synthesis inhibitors are removed. When the stabilizing components are also added to the outer liquid for the dialysis, new stabilizers are always supplied even when the stabilizers are decomposed during the dialysis and, therefore, that is more preferred. That is also applicable to the case where a gel filtration method or an ultrafiltration method is used and the similar effect is able to be achieved when each carrier is equilibrated with a buffer for filtration containing stabilizing components, then the cell extract-containing solution or translation reaction solution containing the stabilizing components is supplied and the filtration is carried out together with addition of the above buffer.

Adding amount of the stabilizing components and time for the stabilizing treatment may be appropriately selected depending upon the type and preparing method of the cell extract-containing solution. An example of the method for the selection is that stabilizing components which were assigned with quantity and type on a test basis are added to a cell extract-containing solution, low-molecular inhibitors are removed after an appropriate period, the resulting cell extract-containing solution after the treatment is separated into soluble and insoluble components by means of centrifugal separation or the like and the product containing less insoluble components is selected. Another preferred method is that a cell-free protein synthesis is carried out using the resulting cell extract-containing solution after the treatment and the product containing (showing) higher protein synthesis activity is selected. Another method is that, when a cell extract-containing solution and a dialysis method are used in the above-mentioned selection method, appropriate stabilizing components are also added to the outer liquid for the dialysis, dialysis is carried for an appropriate period using that and the selection is conducted depending upon the amount of the insoluble components in the resulting cell extract-containing solution or upon the protein synthesis activity, etc. of the resulting cell extract-containing solution.

A specific condition for the stabilization of the cell extract-containing solution selected as such is that, in case the removal step of low-molecular synthesis inhibitors is conducted by a dialysis method, 100 μM to 0.5 mM of ATP, 25 μM to 1 mM of GTP and each 25 μM to 5 mM of 20 kinds of amino acids are added to the wheat embryo extract-containing solution and the outer liquid for the dialysis and dialysis is carried out for 30 minutes to more than 1 hour. With regard to the temperature in case dialysis is carried out, any temperature may be adopted so far as the protein synthesis activity of the cell extract-containing solution is not lost and the dialysis is possible. To be specific, the lowest temperature is the temperature where the solution is not frozen and is usually −10° C. or, preferably, −5° C. while the highest temperature is 40° C. or, preferably, 38° C. which is the limit of the temperature whereby the solution used for the dialysis is not badly affected.

When removal of the low-molecular synthesis inhibitor is carried out after preparing a cell extract-containing solution, it is not necessary that the above stabilizing components are further added to the cell extract-containing solution.

(4) Method for Decreasing the Concentration of a Reducing Agent for the Cell Extract-Containing Solution

When a cell-free protein synthesis is carried out by decreasing the concentration of a reducing agent contained in the cell extract-containing solution, it is possible to prepare protein in such a state that the disulfide bond existing in the molecule of the specific protein is formed. With regard to a method for decreasing the reducing agent in the cell extract-containing solution, there may be used a method where a step for decreasing the reducing agent is carried out in any of the steps until the preparation of a cell extract-containing solution. The reducing agent in terms of the concentration in the finally-prepared cell extract-containing solution is decreased to such a concentration that protein is able to be synthesized in the translation reaction using the said cell extract-containing solution and the intramolecular disulfide bond is able to be formed and retained. With regard to the specific concentration of the reducing agent in the case of dithiothreitol (hereinafter, it may be referred to as “DTT”), its final concentration in the final translation reaction solution prepared from the cell extract-containing solution is decreased to a range of 20 to 70 μM or, preferably, 30 to 50 μM. In the case of 2-mercaptoethanol, the final concentration in the translation reaction solution is decreased to a range of 0.1 to 0.2 mM. In addition, in the case of glutathione/oxidized glutathione, it is decreased so that the final concentration in the translation reaction solution becomes 30 to 50 μM/1 to 5 μM. Concentration of the above-mentioned specific reducing agent is not limited thereto but may be appropriately changed depending upon the type of the protein to be synthesized and upon the type of the cell-free protein synthesis system used.

With regard to a method for selecting the optimum concentration range of a reducing agent, there is no particular limitation and, for example, a method where judgment is done by the effect of the enzyme which catalyzes a disulfide bond exchange reaction may be exemplified. To be specific, translation reaction solutions derived from a cell extract-containing solution to which concentrations of the reducing agent are assigned are prepared and then an enzyme which catalyzes a disulfide bond exchange reaction is added to conduct a synthesis of protein having a disulfide bond in a molecule. As a control experiment, the same protein synthesis is carried out where no enzyme catalyzing a disulfide bond exchange reaction is added to the translation reaction solution which is the same as above. Soluble components of protein synthesized here are separated by, for example, means of a centrifugal separation. A reaction solution in which the soluble components are not less than 50% (solubilizing rate: 50%) of the whole and the soluble components increase as a result of addition of the enzyme catalyzing the disulfide bond exchange reaction is able to be judged to be suitable as a reaction solution which synthesizes the protein wherein the intramolecular disulfide bond is retained. Further, among the concentration range of the reducing agent selected by the effect of the enzyme catalyzing the above disulfide bond exchange reaction, the concentration of the reducing agent where the amount of the synthesized protein is biggest is able to be selected as the more preferred concentration range.

With regard to a specific method for decreasing the reducing agent, there may be used a method where a cell extract-containing solution containing no reducing agent is prepared and a reducing agent is added together with components necessary for a cell-free protein synthesis system thereto so as to give the above-mentioned concentration range, a method where a reducing agent is removed from a translation reaction solution derived from a cell extract-containing solution so as to give the above-mentioned concentration range, etc. A reducing condition of a high degree is necessary for a cell extract-containing solution for a cell-free protein synthesis in conducting the extraction and, therefore, a method where a reducing agent is removed from the solution after extraction is easier. With regard to a method for removing the reducing agent from a cell extract-containing solution, a method where a carrier for gel filtration is used, etc. may be exemplified. To be specific, a method where Sephadex G-25 column is previously equilibrated with an appropriate buffer containing no reducing agent and then a cell extract-containing solution is passed therethrough, etc. may be exemplified.

(5) Preparation of a Translation Reaction Solution

To the cell extract-containing solution which is prepared as such are added nuclease inhibitor, various ions, substrate, energy source, etc. necessary for the synthesis of protein (hereinafter, they may be referred to as “additives to the translation reaction solution”), mRNA encoding a specific protein which is to be a translation template and a stabilizer containing at least one component selected from a group consisting of inositol, trehalose, mannitol and sucrose-epichlorohydrin copolymer whereupon a translation reaction solution is prepared. Adding concentration of each component is able to be achieved by a compounding ratio which has been known per se.

Specific examples of the additives to the translation reaction solution are amino acid to be used as a substrate, energy sources, various ions, buffer, ATP regenerating system, nuclease inhibitor, tRNA, reducing agent, polyethylene glycol, 3′,5′-cAMP, folate and antibacterial agent. With regard to the concentrations, it is preferred to add in such a manner that ATP, GTP and each of 20 kinds of amino acids are made contained in amount ranges of 100 μM to 0.5 mM, 25 μM to 1 mM and 25 μM to 5 mM, respectively. They may be used after appropriately selected and combined depending upon the translation reaction system. To be specific, when a wheat embryo extract solution is used as a cell extract-containing solution, an example is that 20 mM of HEPES-KOH (pH: 7.6), 100 mM of potassium acetate, 2.65 mM of magnesium acetate, 0.380 mM of spermidine (manufactured by Nakarai Tesk), each 0.3 mM of 20 kinds of L-amino acids, 4 mM of dithiothreitol, 1.2 mM of ATP (manufactured by Wako Pure Chemical), 0.25 mM of GTP (manufactured by Wako Pure Chemical), 16 mM of creatinephosphoric acid (manufactured by Wako Pure Chemical), 1 U/μl of RNase inhibitor (manufactured by Takara) and 0.5 μg/l of creatine kinase (manufactured by Roche) are added and, after they are well dissolved, 1 μg of a translation template mRNA (Ω GFP) is added thereto.

Here, with regard to mRNA, anything may be used so far as it has such a structure that a thing encoding protein which is able to be synthesized in a cell-free protein synthesis system is linked at a downstream area of a sequence recognizing an appropriate RNA polymerase and a sequence having a function of activating the translation. An example of the sequence which is recognized by an RNA polymerase is a T3 or T7 RNA polymerase promoter. In preparing a protein chip, library or the like using a reagent for the cell-free protein synthesis of the present invention, it may be appropriately selected depending upon each object. With regard to a sequence which enhances the translation activity in a cell-free protein synthesis system, that having a structure where Ω sequence or the like is linked to a 5′-upstream side of the coding sequence is preferably used.

(6) Method for Freeze-Drying

With regard to a freeze-drying, a cell extract-containing solution, a specific translation template and appropriately selected additives for the translation reaction solution are mixed and a freeze-drying treatment is carried out. An embodiment where selected additives for the translation reaction solution such as nuclease inhibitor, various ions, substrate and energy source are made into a kit separately and it is supplementally added to a protein synthesis reaction system for each use is preferred as well.

Combinations of the embodiment as such are as follows.

Type 1: Cell extract-containing solution, specific translation template and additives for translation reaction solution are uniformly mixed and made into a freeze-dried preparation.

Type 2: Cell extract-containing solution, specific translation template and additives for translation reaction solution are uniformly mixed and made into a freeze-dried preparation and additives for translation reaction solution are made into a kit while the additives for translation reaction solution are added to the upper layer of the cell upon the protein synthesis reaction.

Type 3: Cell extract-containing solution and specific translation template together with additives for translation reaction solution are layered as the lower layer and the upper layer of the cell, respectively and then they are made into a freeze-dried preparation.

Type 4: In addition to the Type 3, additives for translation reaction solution are further made into a kit and, during the protein synthesis reaction, the additives for translation reaction solution are added to the upper layer.

Type 5: Cell extract-containing solution and specific translation template are mixed and made into a freeze-dried preparation while additives for translation reaction solution are made into a kit and the additives for translation reaction solution are added to the upper layer.

A method for the freeze-drying may be appropriately selected from commonly-used methods which have been known per se and, to be specific, it may be carried out in such a manner that, for example, a quick freezing is conducted by liquid nitrogen, reduced pressure is resulted by means of a vacuum pump for example and, after that, temperature of a container is gradually raised. Usually, a commercially available freeze-drier is used. It is preferred that temperature, time, etc. for the freeze-drying are in accordance with the directions for use of the freeze-drier used therefor. The freeze-dried preparation of the present invention which is prepared by completion of dehydration as such is very stable at room temperature and, in addition, a cell extract-containing solution prepared by addition of water or the like thereto has a protein synthesis activity which is favorably comparable with the case of being preserved at an extremely low temperature. Substitution of the air in the container for preservation with nitrogen gas is more preferred as a method for preservation of the said preparation. It is preferred in the present invention that concentration of the cell extract-containing solution during the preparation before freeze-drying is adjusted to 0.1 to 100 mg/ml or, more preferably, 1 to 50 mg/ml. In conducting a freeze-drying, a sample to be charged is placed in a freeze-drier and allowed to stand. After that, the inner temperature of the freeze-drier is cooled down to −30° C. or lower to freeze the sample charged therein. After the freezing, inner area of the freeze-drier is made in vacuo and temperature is raised to such an extent that the charged sample is not melted so as to sublime the water whereupon a primary freeze-drying is carried out. After that, temperature of inner area of the freeze-drier is raised so as to remove the attached water whereupon a secondary drying is carried out.

The container is partitioned in plural sections and there is no particular limitation so far as the above-mentioned translation reaction solutions in the sections as such are not mixed each other. Although there is also no particular limitation for the numbers of the sections, a range of 10 to 200 will be acceptable and particularly preferred ones are those as stipulated by the standards such as 48, 96, 384 and 1536 sections. There is no particular limitation for the material of the container and a commercially available microtiter plate or the like is preferably used. Although there is no particular limitation that, in which well of the container, the reagent for a cell-free protein synthesis containing which translation template is charged, it is preferred that the position (coordinate) of the well is made correspondent to the translation template. When a common automatic dispenser having a tray where a 96-well plate or the like is able to be placed which is commercially available from various companies is used, it is possible that substances necessary for the protein synthesis, translation template and stabilizer are automatically dispensed. An operation for setting the automatic dispenser as such is able to be easily conducted by persons skilled in the art who are usually using automatic dispensers by way of modifying a program.

(7) Method for Preparing a Protein Chip Reagent Utilizing a Cell-Free Protein Synthesis System

Reagent is able to be prepared by containing at least the following elements.

a: A cell extract-containing solution for a cell-free protein synthesis suitable for the volume of the well is added to each of different wells in the container partitioned in plural sections using a pipetteman or the like and/or a channel pipetter of the automatic dispenser.

b: A solution containing necessary amount of a solution containing substances necessary for protein synthesis, translation template and stabilizer is added to each well mentioned in “a” using a pipetteman or the like and/or a channel pipetter of the automatic dispenser.

c: The solution in the well prepared in “b” is made into a freeze-dried preparation using the above freeze-drying method.

If necessary, it is also possible to use a component which was already subjected to the following treatment.

A component where a deliquescent substance in the freeze-dried preparation is decreased.

In a cell extract for a cell-free protein synthesis, a low-molecular protein synthesis inhibitor is removed and/or concentration of a reducing agent is decreased.

(8) Method for the Synthesis of Protein Using a Protein Chip Reagent Utilizing a Cell-Free Protein Synthesis System

The above-prepared protein chip reagent utilizing a cell-free protein synthesis system is dissolved in a dissolving solution where the above-decreased deliquescent substance and water are added so as to give a concentration suitable for the protein synthesis reaction and then poured into each selected system or device which is known per se whereupon protein synthesis is able to be carried out. Examples of the system or device for the protein synthesis are as follows. Thus, a method where, as in the case of a batch method (Pratt, J. M., et al., Transcription and Translation, Hames, 179-209, B. D. &Higgins, S. J., eds., IRL Press, Oxford (1984)), a translation reaction solution in which a protein chip reagent of the present invention utilizing the cell-free protein synthesis system is dissolved is kept at an appropriate temperature; a continuous cell-free protein synthesis system where amino acids, energy source, etc. necessary for the cell-free protein synthesis system are continuously supplied to reaction system (Spirin, A. S., et al., Science, 242, 1162-1164 (1988)); a dialysis method (Kigawa, et al., The 21st Meeting of the Japan Molecular Biology Association, WID 6); a method where a solution containing amino acids, energy source, etc. necessary for a cell-free protein synthesis system is layered on a translation reaction solution (a multilayered method; Laid-Open Patent No. WO 02/24939 A1); etc.

When a protein chip reagent utilizing a cell-free protein synthesis system where concentration of a reducing agent is decreased is used, a reducing agent is also adjusted to the same concentration even for a solution which provides amino acids, energy source, etc. necessary for a cell-free protein synthesis system. When the translation reaction is carried out in the presence of an enzyme which catalyzes a disulfide bond exchange reaction, protein in which intramolecular disulfide bond is retained is able to be synthesized in high efficiency. An example of the enzyme which catalyzes a disulfide bond exchange reaction is a protein disulfide isomerase. Adding amount of such an enzyme to the above-mentioned cell-free translation system may be appropriately selected depending upon the type of the enzyme. To be specific in a solution containing a cell extract for a cell-free protein synthesis prepared from a wheat embyo, when a protein disulfide isomerase is added to a translation reaction solution containing 20 to 70 or, preferably, 30 to 50 μM of DTT as a reducing agent, it is added within such an extent that its final concentration in the translation reaction solution is made within a range of 0.01 to 10 μM or, preferably, 0.5 μM. With regard to the stage for the addition, it is preferred to add prior to the start of the translation reaction in view of efficiency for the formation of a disulfide bond.

(9) Preparation of a Protein Library

In the reagent and/or the kit of the present invention, different kind type of translation template is contained in each a solution are freeze-dried in different wells of a container which is partitioned into plural sections whereupon a kit for preparing a protein library is prepared. Method for preparing a translation reaction solution, adding amount of mRNA which is a translation template, etc. are the same as those mentioned above already. In addition, container and method for freeze-drying are the same as those mentioned above already.

When a buffer having a concentration suitable for a cell-free protein synthesis reaction is added to the reagent for protein library according to the present invention so that the reagent is dissolved therein and kept at an appropriate temperature, two or more kinds of proteins are able to be synthesized at the same time and a protein library can be prepared. Method for the translation reaction is the same as those mentioned above already.

The protein library which is prepared using a reagent for a protein library according to the present invention may be used, for example, as a protein chip. Especially when a mechanized protein chip production is carried out, it is preferred to use a microtiter plate meeting the standards for a protein chip manufacturing device as a container for a kit of the present invention.

(10) Embodiments of Protein Chip Reagent

A translation template contained in the translation reaction solution has a structure of adding a modification for fixation to protein encoded thereby and two or more translation reaction solutions each containing different translation template are freeze-dried in a container partitioned into plural sections and in each different well where a substance having an affinity to the substance which is added as a result of the above-mentioned modification for fixation is coated on bottom of the well and/or carrier in the well whereupon a protein chip reagent is prepared.

In an embodiment of protein chip, it means a chip where two more kinds of protein are separately fixed on an appropriate base (surface of a well) or a chip which is present in a well. The protein chip is used for analysis (hereinafter, it may be referred to as “proteome analysis”) of bonding property or interacting property of protein with protein, nucleic acid, low-molecular compound or sugar chain, etc.

When a buffer having a concentration suitable for a cell-free protein synthesis reaction is added to the protein chip reagent according to the present invention so that the reagent is dissolved therein and kept at an appropriate temperature, several kinds of proteins are able to be synthesized at the same time and, since the synthesized proteins are bonded to the bottom of the well or are present in the well, it is possible to prepare a protein chip. A method for the translation reaction is the same as that mentioned hereinabove already.

(11) Test Method for the Interacting Substances

A method for testing the interacting substances is able to be carried out by including at least the following steps.

a: a freeze-dried reagent for a cell-free protein synthesis is dissolved upon each use; and

b: after dissolving the reagent for a cell-free protein synthesis, conditions for translation reaction of protein are adjusted, synthesis of specific protein is carried out by the above-mentioned method and the said protein is expressed in a well.

If necessary or depending upon the type of the sample, the following steps are added.

c: necessary amount of the sample is added to a well and interaction of the synthesized specific protein with a sample is carried out; and

d: on the basis of the result of the interaction, a substance which interacts with the specific protein is judged from the sample either qualitatively or quantitatively.

In the above-mentioned protein chip reagent and a test method for the interacting substance with a specific protein, protein which is synthesized by a cell-free protein synthesis system if desired is modified for fixation and, further, it is possible to use a well and/or a carrier which is coated with a substance having an affinity for a substance added by a modification for the said fixation to a carrier on the surface of a well and/or in a well.

Examples of a combination of a modification for fixation with a substance having affinity for a substance being added by modification for fixation are protein bonded to biotin such as avidin or streptoavidin/biotin, protein bonded to maltose/maltose, G protein/guanine nucleotide, polyhistidine peptide/metal such as nickel or cobalt, glutathione-S-transferase/glutathione, protein bonded to DNA/DNA, antibody/antigen molecule (epitope), calmodulin/peptide bonded to calmodulin, protein bonded to ATP/ATP or estradiol receptor protein/estradiol and other various receptor protein/ligand thereof. With regard to a carrier, beads, Sephadex resin, etc. may be exemplified although there is no particular limitation provided that the synthetic protein is able to be bonded to the surface of a carrier.

With regard to a method for modification for fixation to protein encoded by translation template, any method will be acceptable so far as it is a method where modification for fixation is possible to protein which is synthesized in a cell-free protein synthesis system. To be specific, there may be used a method where synthesis of protein is carried out using a translation reaction solution in which tRNA bonded to a substance to be added by means of modification for fixation (hereinafter, that may be referred to as “substance for fixation”) is added; a method where synthesis of protein is carried out using a product where a sequence encoding a substance for fixation is added to a translation template in case polypeptide is used as the substance; a method where a substance for fixation is directly added to C terminal of protein by a nucleic acid derivative such as puromycin in a protein synthesis system (Japanese Laid-Open patent Ser. No. 11/322,781); etc. Here, it is preferred to insert a spacer having a suitable length between a substance for fixation and mRNA encoding protein. With regard to a spacer, a macromolecular substance such as polyethylene and polyethylene glycol may be used and, preferably, polyethylene glycol is used.

With regard to a method for bonding a substance (which specifically bonds to a substance for fixation) to a well, a method which has been known per se may be used and, to be specific, a method using, for example, tannic acid, formalin, glutaraldehyde, pyruvic aldehyde, bis-diazotized benzisone, toluene-2,4-diisocyanate, amino group, carboxyl group or hydroxyl group or amino group, etc. may be used.

With regard to a container used, the above-mentioned one may be used and that may be appropriately selected depending upon a method for the detection used in proteome analysis. To be specific, that which is made of a material such as plastic, polycarbonate, complex carbohydrate, acrylic resin, nitrocellulose, glass or silicon may be exemplified. When a surface plasmon method (Cullen, D. C., et al., Biosensors, 3(4), 211-225 (1987-88)) is used for the proteome analysis, that where thin membrane of metal such as gold, silver and platinum is constituted on a transparent substrate such as glass is used.

A translation template contained in a translation reaction solution may be appropriately selected depending upon the purpose of the proteome analysis using a protein chip prepared by the protein chip synthesis kit of the present invention. To be specific, when it is used for the analysis of function of protein, mRNA having a sequence encoding a functional protein is selected. Its examples are ion channel, transporter, growth factor, hydrolase, synthase, oxidation-reduction enzyme, proteinous inhibitor, physiologically active peptide, peptidergic toxin, receptor such as that of a G protein conjugation type, ligand and antibody. With regard to a sample to be measured, although there is no particular limitation so far as it is a substance which interacts with specific protein and/or translation template for the said specific protein, its examples are protein, nucleic acid, low-molecular compound and sugar chain.

There is no particular limitation for the fact that a cell-free protein synthesis reagent containing what translation template is placed in which well of a container although it is preferred that the position (coordinate) of a well corresponds to the translation template. Incidentally, a method for the freeze-drying is the same as that mentioned above already.

With regard to a method for a qualitative or quantitative determination of a substance which interacts with a specific protein, there is no particular limitation so far as it is a method being able to detect an interaction of the sample with the specific protein. To be specific, when analysis is carried out by means of an enzyme-linked immunosorbent assay (ELISA) (Crowthjer, J. R., Methods in Molecular Biology, 42, (1995)), a microtiter plate made of plastic which is usually used in ELISA is preferred. When a surface plasmon resonance method (Cullen, D. C., et al., Biosciences, 3(4), 211-225 (1987-88)) is used, that where thin film of metal such as gold, silver or platinum is constituted on a transparent substrate such as glass is preferred. When an evanescent field molecular imaging method (Funatsu, T., et al., Nature, 374, 555-559 (1995)) is used, a transparent substance such as glass is preferred and, more preferably, that which is made of quartz glass is used. When a fluorescence imaging analysis method is used, it is also possible to use nitrocellulose membrane filter or Nylon membrane which is usually used for fixation of protein or the like or microtiter plate made of plastic, etc. may be used as well. It is also possible that a substrate is labeled, a specific protein synthesized by a cell-free protein synthesis system is labeled and the interacting substance is traced either qualitatively or quantitatively using the said label as a marker. With regard to a labeling means at that time, common means such as heavy hydrogen, radioisotope, fluorescent substance and chromogen substance may be exemplified. It is also possible to conduct a qualitative and quantitative judgment of interaction in a substance which interacts with a translation template of a specific protein from the expressed amount of the specific protein expressed in a cell-free protein synthesis system by the presence of a substance which interacts with a translation template of a specific protein or from the fact whether or not the labeled specific protein is expressed.

The present invention will now be illustrated in detail by way of the following Examples although the present invention is not limited by those Examples.

### EXAMPLE 1

Protein Synthesis Using a Freeze-Dried Reagent of a Translation Reaction Solution Containing a Translation Template

(1) Preparation of a Wheat Embryo Extract

Seeds of wheat (non-disinfected) of chihoku variety produced in Hokkaido were added at a rate of 100 g per minute to a mill (Rotor Speed Mill Pulverisette 14 Type manufactured by Fritsch) and gently disintegrated at 8,000 rpm. A fraction (mesh size: 0.7 to 1.00 mm) containing embryo having a germination ability was recovered using a sieve, subjected to a floatation using a mixture of carbon tetrachloride and cyclohexane (ratio by volume of carbon tetrachloride to cyclohexane=2.4:1) to recover a floated fraction containing the embryo having a germination ability, the organic solvents were removed by evaporation at room temperature and impurities such as seed coat being mixed as a result of ventilation at room temperature were removed to give a crude embryo fraction.

After that, embryo was selected from the crude embryo fraction as follows utilizing the difference in color using a belt-type color selector BLM-300 K (manufactured by K. K. Anzai Seisakusho; distributor: K. K. Anzai Sogyo). The color selector is a device equipped with a means for irradiating the light to the crude embryo fraction, a means for detecting the reflected light and/or transmitted light from the crude embryo fraction, a means for comparing the detected value with the standard value and a means for the selection and removal of the thing which is out of or within the standard value.

The crude embryo fraction was supplied onto a beige belt of the color selector at the rate of 1,000 to 5,000 grains/cm², light from a fluorescent lamp is irradiated to the crude embryo fraction on the belt and the reflected light was detected. Conveying speed of the belt was made 50 m/minute. A monochrome CCD line sensor (2048 pixels) was used as a light-receiving sensor.

Firstly, in order to remove black components (seed coat, etc.) from the embryo, a standard value was set up between luminance of the embryo and luminance of the seed coat and those which were out of the standard value were removed by suction. After that, in order to select the endosperm, a standard value was set up between luminance of the embryo and luminance of the endosperm and those which were out of the standard value were removed by suction. The suction was carried out using 30 suction nozzles (one suction nozzle for each cm of the length) which were placed at the positions of about 1 cm above the conveying belt.

As a result of repetition of such a method, the embryo was selected until purity of the embryo (rate by weight of the embryo contained in 1 g of a sample) became 98% or more.

The resulting wheat embryo fraction was suspended in distilled water of 4° C. and washed using an ultrasonic cleaner until the washing was no longer turbid. After that, it was suspended in a 0.5 vol. % solution of Nonidet P40 (manufactured by Nakarai Tesk), washed using an ultrasonic cleaner until the washing was no longer turbid to give wheat embryo and the following operation was conducted at 4° C.

To the washed wet embryo were added 2-fold by volume of an extracting solvent (80 mM of HEPES-KOH, pH 7.8, 200 mM of potassium acetate, 10 mM of magnesium acetate and 8 mM of dithiothreitol (each 0.6 mM of 20 kinds of L-amino acids may be added thereto)) and a limited disintegration of embryo was carried out for three times at 5,000-20,000 rpm for 30 seconds using a Waring blender. A supernatant liquid prepared by centrifugal separation of the above-prepared homogenate at 30,000×g for 30 minutes using a high-speed centrifugal separator was subjected to a centrifugal separation once again under the same condition and the supernatant liquid was prepared. The resulting sample did not show deterioration of activity upon a long-term preservation at not higher than −80° C. The resulting supernatant liquid was passed through a filter having a pore size of 0.2 μm (New Stella Disk 25, manufactured by Kurabo) whereupon sterilization by filtration and removal of contaminated fine dusts were conducted.

After that, the filtrate was subjected to gel filtration using a column of Sephadex G-25 which was previously equilibrated with a solution [40 mM HEPES-KOH (pH 7.8) each being mixed with 100 mM of potassium acetate, 5 mM of magnesium acetate, 8 mM of dithiothreitol and each 0.3 mM of 20 kinds of L-amino acid mixed solution (the amino acids may not be added or they may be labeled amino acids depending upon the object of the synthesis of protein)]. The resulting filtrate was subjected to a centrifugal separation again at 30,000×g for 30 minutes and concentration of the recovered supernatant liquid was adjusted to such an extent that A_{260 nm} was 90 to 150 (A₂₆₀/A₂₈₀=1.4-1.6).

To the resulting cell extract-containing solution for the synthesis of protein were added 20 mM of HEPES-KOH (pH 7.6), 100 mM of potassium acetate, 2.65 mM of magnesium acetate, 0.380 mM of spermidine (manufactured by Nakarai Techtonics), each 0.3 mM of 20 kinds of L-amino acids, 4 mM of dithiothreitol, 1.2 mM of ATP (manufactured by Wako Pure Chemicals), 0.25 mM of GTP (manufactured by Wako Pure Chemicals), 16 mM of creatine phosphate (manufactured by Wako Pure Chemicals), 1 U/μl of RNase inhibitor (manufactured by Takara) and 0.5 μg/l of creatine kinase (manufactured by Roche), whereupon a translation reaction solution material was prepared.

(2) Addition of Translation Template

mRNA (Ω GFP) which is to be a translation template was subjected to transcription using SP6 RNA polymerase (manufactured by Toyobo) where an omega (ω) sequence of tobacco mosaic virus (TMV) which is a translation initiation reaction sequence connected to an SP6 promoter sequence and also a pladmid GFP/pEU containing GFP gene DNA connected to 3′-downstream (WO 01/27260) were used as templates, the resulting RNA was extracted with phenol/chloroform, precipitated with ethanol and used after purification with a Nick Column (manufactured by Amersham Pharmacia Biotech). The mRNA (1 μg) was added to the translation reaction solution material manufactured in Example 1 to prepare a translation reaction solution.

(3) Freeze-Dried Preparation

The above-mentioned translation reaction solution to which the translation template was added was dialyzed against an outer liquid for dialysis to give a translation reaction solution of a type where a deliquescent substance was decreased (concentration of potassium acetate was 0 to 50 mM).

After the dialysis, each translation reaction solution was frozen with liquid nitrogen and dehydrated by an operation for 3 hours using a freezer-drier (Freeze Dry System Freezone 4.5; manufactured by Labconco) in accordance with the direction for use attached thereto. A powdery sample prepared as such was tightly sealed being filled with nitrogen so that the components were not chemically changed and preserved at 4° C.

(4) Translation Reaction and Analysis of Amount of the Synthesized Protein

The reagent for a cell-free protein synthesis which was made into a freeze-dried preparation as above was dissolved in 40-50 μl of a 100 mM magnesium solution. As a control, a preparation where 1 μg of the translation template mRNA mentioned in the above-mentioned step (3) was added to a translation reaction solution material liquid prepared in the above-mentioned step (1) was prepared. Those translation reaction solutions were subjected to a dialysis system using an outer liquid for dialysis (20 mM of HEPES-KOH, pH 7.6, 100 mM of potassium acetate, 2.65 mM of magnesium acetate, 4 mM of dithiothreitol, 1.2 mM of ATP, 0.25 mM of GTP, 16 mM of creatine phosphate, 0.5 mg/ml of creatine kinase, 0.380 mM of spermidine, 20 kinds of L-amino acids (each 0.3 mM) and 0.005% of NaN₃) in an amount of 10-fold by volume of the reaction solution and protein synthesis reaction was carried out at 26° C. for 48 hours. Each 5 μl of the translation reaction solution were recovered every 6, 12, 24 and 48 hours and used for the measurement of amount of the synthesized protein. As to a permeable membrane, Spectra/Pore 6 (Spectrum Laboratories, Inc., CA, U.S.A.) having a removing molecular weight of 50,000 was used.

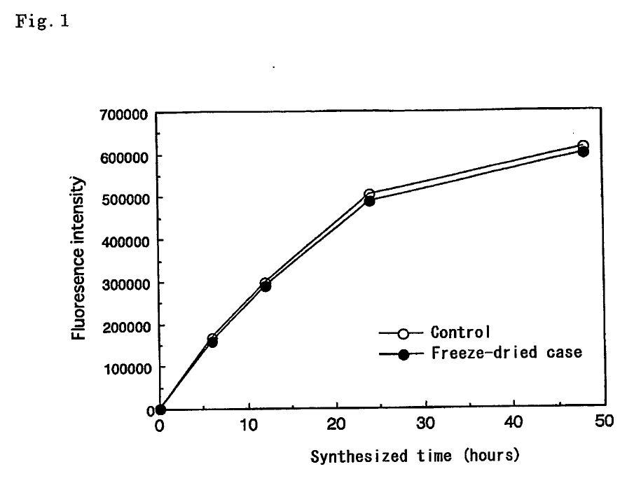

Synthesized amount of protein in each translation reaction solution after completion of the reaction was measured by means of fluorescence amount of GFP. The fluorescence amount of GFP was quantified from fluorescence intensity using TD-360 Mini-Fluorometer manufactured by Turner Design according to the attached instruction. The results are shown in FIG. 1.

As will be apparent from FIG. 1, even when freeze-drying was conducted by addition of a translation template (● in the graph), there was noted the same synthetic activity as in the case where the translation solution was not freeze-dried and the translation template was added later (◯ in the graph) and it was noted that, even when the reaction time was longer, the synthetic activity was kept high.

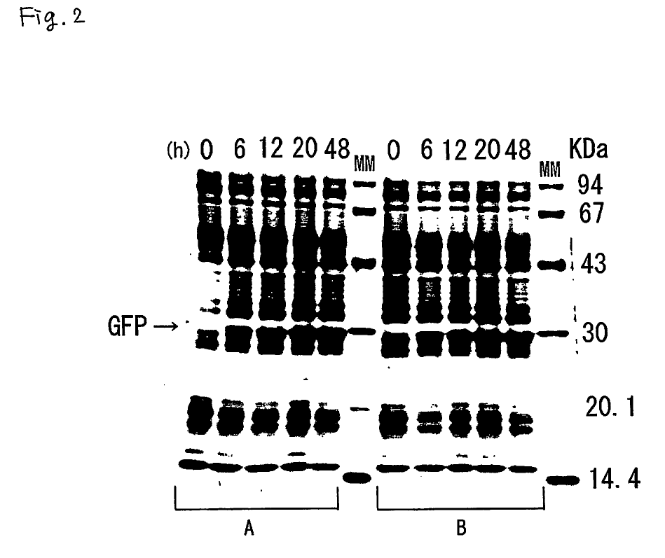

The result when the translation reaction solution obtained by the same experiment was separated by an SDS-polyacrylamide gel electrophoresis and the gel was stained with Coomassie-Brilliant Blue is shown in FIG. 2. Columns in FIG. 2A show the reaction products of a synthesis system where the translation reaction solution was not freeze-dried and the translation template was added later while columns in FIG. 2B show the reaction products of a synthesis system where the translation reaction solution to which the translation template was added was freeze-dried. As will be apparent from FIG. 2B, the translation reaction solution which was freeze-dried in a state where the translation template was added showed the same protein synthesis activity as in the case where the translation reaction solution which was not freeze-dried was used.

### EXAMPLE 2

Simultaneous Synthesis of Polyclonal Protein

Each 30 μg of 96 kinds of translation template mRNA were added to the translation reaction solution prepared by the same manner as in Example 1 and each 50 μl thereof were placed in each well of a 96-well microtiter plate. That was freeze-dried and preserved by the same manner as in Example 1. Each 40 to 50 μl of a 100 mM potassium acetate solution were placed to and dissolved in each well containing the resulting reagent for a cell-free protein synthesis. After that, to each solution were added 4 μCi of ¹⁴C-leucine (100 mCi/mmol) per ml of the translation reaction solution. A solution comprising 30 mM of Hepes-KOH (pH 7.6), 95 mM of potassium acetate, 2.65 mM of magnesium acetate, 2.85 mM of dithiothreitol, 1.2 mM of ATP, 0.25 mM of GTP, 16 mM of creatine phosphate, 0.380 mM of spermidine and 20 kinds of L-amino acids (0.3 mM) as a solution for supplying of substrate and energy (hereinafter, that will be referred to as “supplying solution”) containing amino acids, energy source and other ions, etc. necessary for the reaction was layered on the upper layer of the solution without disturbing the interface.

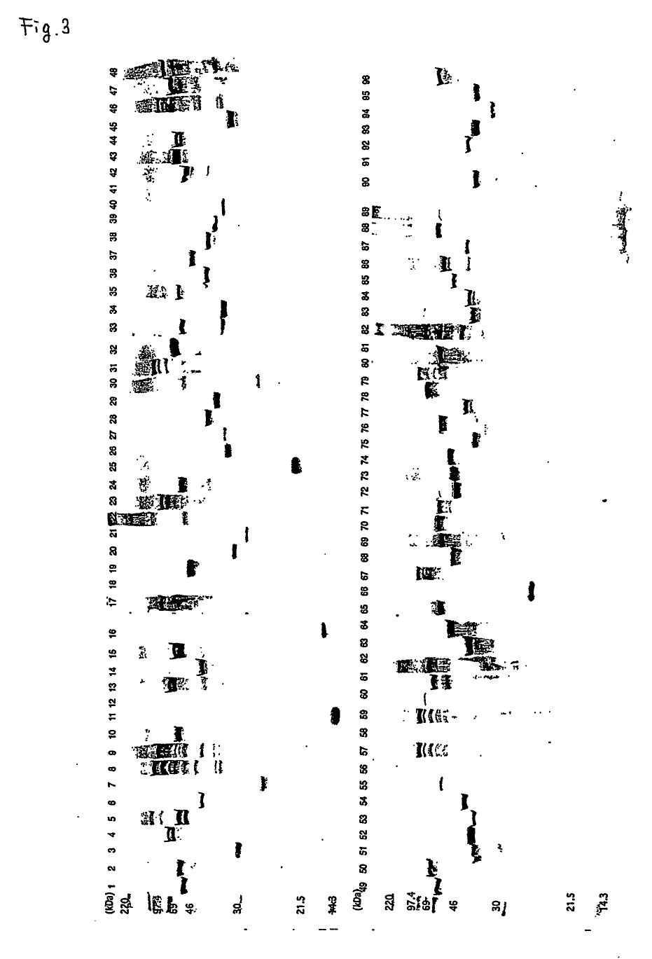

Those were made to react at 26° C. for 20 hours and 5 μl of the solution after completion of the reaction was subjected to an autoradiography according to a method mentioned in Endo, Y., et al., J. Biotech., 25, 221-230 (1992) and Endo, Y., et al., Proc. Natl. Acad. Sci., USA, 97, 559-564 (2000). The result is shown in FIG. 3. As will be apparent from the drawing, most of the 96 kinds of translation templates were translated in each well of one microtiter plate whereupon protein was synthesized.

### INDUSTRIAL APPLICABILITY

According to the reagent of the present invention for a cell-free protein synthesis where a translation reaction solution containing a translation template is made into a freeze-dried preparation, it is now possible to synthesize a specific protein in a high efficiency only by means of adding and dissolving a solution containing a deliquescent substance such as potassium acetate which is decreased for freeze-drying followed by keeping it at an appropriate temperature. According to such a technique, there is provided a kit for preparing a protein library containing a synthetic reagent containing freeze-dried different translation templates in each well of a microtiter plate and there is also provided a kit for preparing a protein chip where a structure of binding to the bottom of a synthetic container is given to the synthesized protein. As such, the present invention is particularly useful for a high throughput analysis such as function of protein handling many clones or for a test using a specific protein.

1. A protein chip reagent utilizing a cell-free protein synthesis system which comprises the following elements:
a: a solution containing cell extract for a cell-free protein synthesis comprising a wheat embryo extract wherefrom endosperm components and low-molecular protein synthesis inhibitors are substantially removed is added to each different well of a container which is partitioned in plural sections;
b: at least the substances necessary for protein synthesis containing substrate and energy source and a specific translation template are added to the well mentioned in “a”; and
c: the solution in the well mentioned in “b” is freeze-dried preparation (by freeze-drying).

2. A protein chip reagent utilizing a cell-free protein synthesis system which comprises the following elements:
a: a solution containing cell extract for a cell-free protein synthesis comprising a wheat embryo extract wherefrom endosperm components and low-molecular protein synthesis inhibitors are substantially removed is added to each different well of a container which is partitioned in plural sections;
b: at least the substances necessary for protein synthesis containing substrate and energy source and a specific translation template are added to the well mentioned in “a”;
c: the solution in the well mentioned in “b” is freeze-dried preparation (by freeze-drying); and
d: amount of a deliquescent substance in the freeze-dried preparation in the well mentioned in “c” is 0.01 part by weight or less to 1 part by weight of the protein in the said freeze-dried preparation.

3. A protein chip reagent utilizing a cell-free protein synthesis system which comprises the following elements:
a: a solution containing cell extract for a cell-free protein synthesis comprising a wheat embryo extract wherefrom endosperm components and low-molecular protein synthesis inhibitors are substantially removed is added to each different well of a container which is partitioned in plural sections;
b: at least the substances necessary for protein synthesis containing substrate and energy source and a specific translation template are added to the well mentioned in “a”;
c: the solution in the well mentioned in “b” is freeze-dried preparation (by freeze-drying);
d: amount of a deliquescent substance in the freeze-dried preparation in the well mentioned in “c” is 0.01 part by weight or less to 1 part by weight of the protein in the said freeze-dried preparation; and
e: different kind type of translation template is contained in each the solution mentioned in “b” and makes two or more kinds of proteins synthesizable in each different well of the container which is partitioned in plural sections.

4. A protein chip reagent utilizing a cell-free protein synthesis system which comprises the following elements:
a: a solution containing cell extract for a cell-free protein synthesis comprising a wheat embryo extract wherefrom endosperm components and low-molecular protein synthesis inhibitors are substantially removed is added to each different well of a container which is partitioned in plural sections;
b: at least the substances necessary for protein synthesis containing substrate and energy source and a specific translation template are added to the well mentioned in “a”;
c: the solution in the well mentioned in “b” is freeze-dried preparation (by freeze-drying);
d: amount of a deliquescent substance in the freeze-dried preparation in the well mentioned in “c” is 0.01 part by weight or less to 1 part by weight of the protein in the said freeze-dried preparation;
e: different kind type of translation template is contained in each the solution mentioned in “b” and makes two or more kinds of proteins synthesizable in each different well of the container which is partitioned in plural sections; and
f: protein synthesized from the translation template is modified for fixation and is also coated with a substance having affinity to a substance added by the said modification for fixation to a surface in the well and/or a carrier in the well.

5. The protein chip reagent utilizing a cell-free protein synthesis system according to claim 4, wherein the modification for fixation is at least one which is selected from making into avidin, biotin, streptoavidin and His tag.

6. A kit for a cell-free protein synthesis containing the protein chip reagent utilizing a cell-free protein synthesis system mentioned in claim 1.

7. A test method for an interacting substance with a specific protein translated from a specific translation template containing the following elements where a reagent mentioned in claim 1 is used,
(1) a protein chip reagent utilizing a cell-free protein synthesis system is dissolved upon each use;
(2) after dissolving, conditions for a protein translation reaction are regulated to synthesize a specific protein;
(3) a substance to be detected is added and it is confirmed whether the interaction with the specific protein which is synthesized upon each use takes place; and
(4) the interacted substance is judged either qualitatively or quantitatively using a marker.

8. A kit for a cell-free protein synthesis containing the protein chip reagent utilizing a cell-free protein synthesis system mentioned in claim 2.

9. A kit for a cell-free protein synthesis containing the protein chip reagent utilizing a cell-free protein synthesis system mentioned in claim 3.

10. A kit for a cell-free protein synthesis containing the protein chip reagent utilizing a cell-free protein synthesis system mentioned in claim 4.

11. A kit for a cell-free protein synthesis containing the protein chip reagent utilizing a cell-free protein synthesis system mentioned in claim 5.

12. A test method for an interacting substance with a specific protein translated from a specific translation template containing the following elements where a reagent mentioned in claim 2 is used,
(1) a protein chip reagent utilizing a cell-free protein synthesis system is dissolved upon each use;
(2) after dissolving, conditions for a protein translation reaction are regulated to synthesize a specific protein;
(3) a substance to be detected is added and it is confirmed whether the interaction with the specific protein which is synthesized upon each use takes place; and
(4) the interacted substance is judged either qualitatively or quantitatively using a marker.

13. A test method for an interacting substance with a specific protein translated from a specific translation template containing the following elements where a reagent mentioned in claim 3 is used,
(1) a protein chip reagent utilizing a cell-free protein synthesis system is dissolved upon each use;
(2) after dissolving, conditions for a protein translation reaction are regulated to synthesize a specific protein;
(3) a substance to be detected is added and it is confirmed whether the interaction with the specific protein which is synthesized upon each use takes place; and
(4) the interacted substance is judged either qualitatively or quantitatively using a marker.

14. A test method for an interacting substance with a specific protein translated from a specific translation template containing the following elements where a reagent mentioned in claim 4 is used,
(1) a protein chip reagent utilizing a cell-free protein synthesis system is dissolved upon each use;
(2) after dissolving, conditions for a protein translation reaction are regulated to synthesize a specific protein;
(3) a substance to be detected is added and it is confirmed whether the interaction with the specific protein which is synthesized upon each use takes place; and
(4) the interacted substance is judged either qualitatively or quantitatively using a marker.

15. A test method for an interacting substance with a specific protein translated from a specific translation template containing the following elements where a reagent mentioned in claim 5 is used,
(1) a protein chip reagent utilizing a cell-free protein synthesis system is dissolved upon each use;
(2) after dissolving, conditions for a protein translation reaction are regulated to synthesize a specific protein;
(3) a substance to be detected is added and it is confirmed whether the interaction with the specific protein which is synthesized upon each use takes place; and
(4) the interacted substance is judged either qualitatively or quantitatively using a marker.
